(19) United States
(12) Patent Application Publication
(10) Pub. No.: US 2002/0006661 A1
(43) Pub. Date: Jan. 17, 2002

(54) Controlling gene expression in living cells

(75) Inventors: Michael R. Green, Boylston, MA (US); Geoff Werstuck, Ancaster (CA)

Correspondence Address: J. PETER FASSE, Fish & Richardson P.C., 225 Franklin Street, Boston, MA, 02110-2804, US

(73) Assignee: University of Massachusetts, a Massachusetts corporation

(21) Appl. No.: 09/824,358

(22) Filed: Apr. 2, 2001

Related U.S. Application Data
(63) Continuation of application No. 09/169,446, filed on Oct. 8, 1998, now abandoned.

(51) Int. Cl. C12N 5/06; A61K 31/00; C12Q 1/00; C07H 21/04

(52) U.S. Cl. 435/325; 435/4; 536/23.1; 514/1

(57) ABSTRACT

Methods for controlling expression of a gene in a living cell are disclosed. In general, the methods include contacting the 5′ untranslated region (5′ UTR) of an RNA in the cell with a cell permeable, small molecule. In some embodiments of the invention, the method includes providing an aptamer that binds specifically to the cell permeable, small molecule; incorporating the aptamer into a region of a gene, which region encodes a 5′ UTR of an RNA; and contacting the cell-permeable, small molecule with a cell that contains the gene. The cell-permeable, small molecule enters the cell and binds specifically to the aptamer sequence in the 5′ UTR of RNA molecules transcribed from the gene. This binding specifically inhibits translation of the RNA molecules to which the cell permeable, small molecule is bound, thereby controlling expression of the gene.

### FIELD OF THE INVENTION

The invention relates to biochemistry, molecular biology, cell biology, medicine, and gene therapy.

### BACKGROUND OF THE INVENTION

A method commonly known as “in vitro selection” (Ellington et al., Nature 346:818-822 (1990), “in vitro evolution” (Joyce, Gene 82:83-87 (1989), or “SELEX” (Selective Evolution of Ligands by Evolution) Tuerk et al., Science 249:505-510 (1990) allows the screening of large random pools of nucleic acid molecules for a particular functionality. This technique has been used to screen for functionalities such as binding to small organic molecules (Famulok et al., Am. J. Chem. Soc. 116:1698-1706 (1994); Connell et al., Biochemistry 32:5497-5502 (1994); Ellington et al., Nature 346:818-822 (1990)), large proteins (Jellinek et al., Proc. Natl. Acad. Sci. USA 90:11227-11231 (1993); Tuerk et al., Proc. Natl. Acad. Sci. USA 89:6988-6992 (1992); Tuerk et al., Gene 137:33-39 (1993); Schneider et al., J. Mol. Biol. 228:862-869 (1992)); and the alteration or de novo generation of ribozymes (Liu et al., Cell 77:1093-1100 (1994); Green et al., Nature 347:406-408 (1990); Green et al., Science 258:1910-1915 ((1992); Pun et al., Biochemistry 31:3887-3895 (1992); Bartel et al., Science 261:1411-1418 (1993). Functional molecules, known as “aptamers” (from “aptus,” Latin for fit) are selected by column chromatograpy or any other technique of enrichment for the desired function.

For in vitro selection, a pool of oligonucleotides is synthesized with a completely random base sequence flanked PCR primer binding sites. The pool is subjected to the enrichment step, and then selected molecules are amplified in a PCR step. Up to 10¹⁵ different molecules, i.e., every possible permutation of an oligonucleotide containing a 25-base sequence, can be generated in this way and then screened simultaneously. Large numbers of random permutations of longer base sequences can be generated by carrying out the PCR step under mutagenic conditions (Lehman et al., Nature 361:182-185 (1993); Beaudry et al., Science 257:635-641 (1992)).

### SUMMARY OF THE INVENTION

We have discovered that aptamers incorporated into an RNA faithfully bind their ligand in vivo. Based on this discovery, the invention provides methods for controlling expression of a gene in a living cell. In general, the method includes contacting the 5′ untranslated region of an RNA in the cell with a cell permeable, small molecule. In some embodiments of the invention, the method includes providing an aptamer that binds specifically to a cell permeable, small molecule; incorporating the aptamer into a region of a gene, which region encodes a 5′ untranslated region (5′ UTR) of an RNA; and contacting the cell-permeable, small molecule with a cell that contains the gene. The cell-permeable, small molecule enters the cell and binds specifically to the aptamer sequence in the 5′ UTR of RNA molecules transcribed from the gene. This binding specifically inhibits translation of the RNA molecules to which the cell permeable, small molecule is bound, thereby controlling expression of the gene.

The gene whose expression is controlled can be an endogenous gene or a transgene. The cell can be a prokaryotic cell or a eukaryotic cell. In some embodiments, the eukaryotic cell is a mammalian cell. The mammalian cell can be in vivo, e.g., in a human receiving gene therapy. The cell permeable molecule can be administered to the mammal by any suitable route, e.g., topically, parenterally, orally, vaginally, or rectally.

The invention also provides a gene containing an aptamer sequence incorporated into a region of the gene that encodes a 5′ UTR of an RNA. The invention also provides a transgenic cell containing an aptamer incorporated into a region of a gene that encodes a 5′ UTR of an RNA. Preferably, the cell includes an RNA transcript containing the aptamer in the 5′ UTR of the RNA transcript. The cell can contain a cell permeable, small molecule that binds specifically to the aptamer.

The invention also provides a bacterial resistance marker. The marker includes an aptamer sequence operably linked to a bacterial expression control sequence.

The invention also provides a method for determining whether a gene of interest is essential for the survival or growth of a cell. This method is useful in target validation studies. The method includes structurally disrupting or deleting an endogenous gene of interest in a cell; providing an aptamer that binds specifically to a cell permeable, small molecule; incorporating the aptamer into a region of the gene of interest in vitro, which region encodes a 5′ untranslated region of an RNA, thereby producing a controllable gene of interest; introducing the controllable gene of interest into the cell, thereby producing a test cell; and contacting the cell-permeable, small molecule with the test cell, so that the cell-permeable, small molecule enters the test cell and controls expression of the controllable gene of interest.

As used herein, “cell permeable, small molecule” means a molecule that permeates a living cell without killing the cell, and whose molecular mass is about 1,000 Daltons or less.

Unless otherwise defined, all technical and scientific terms used herein have the same meaning as commonly understood by one of ordinary skill in the art to which this invention belongs. In case of conflict, the present application, including definitions will control. All publications, patents, and other references mentioned herein are incorporated by reference.

Although methods and materials similar or equivalent to those described herein can be used in the practice or testing of the present invention, the preferred methods and materials are described below. The materials, methods, and examples are illustrative only and not intended to be limiting. Other features and advantages of the invention will be apparent from the detailed description, and from the claims.

### BRIEF DESCRIPTION OF THE DRAWINGS

FIG. 1 is a tobramycin-binding consensus aptamer nucleic acid sequence, with predicted secondary structure indicated.

FIG. 2 is a kanamycin A-binding consensus aptamer nucleic acid sequence, with predicted secondary structure indicated.

FIGS. 3A-3E are growth curves of E. coli expressing antibiotic aptamers. Overnight cultures of BL-21 cells transformed with plasmids expressing RSETA, tob1, tob3, kan1 or kan3 were diluted 100-fold into medium containing the indicated concentration of aminoglycoside antibiotic. Optical density (660 nm) was measured at fixed intervals over 8 hours of growth at 37° C. FIG. 3A shows data on bacterial growth in the absence of drug. FIG. 3B shows data on bacterial growth in the presence of 10 μM Kanamycin A. FIG. 3C shows bacterial growth in the presence of 10 μM Tobramycin. FIG. 3D shows growth in the presence of 20 μM Kanamycin A. FIG. 3E shows bacterial growth in the presence of 20 μM Tobramycin.

FIG. 4. is a histogram showing percent translation of mRNA in a wheat germ in vitro translation system containing 0 (RSETA) or 3 copies of the tob aptamer cloned into the 5′ UTR of RSETA (tob3-RSETA) and 0, 30 or 60 μM tobramycin or kanamycin A. Protein products were analyzed by SDS-PAGE and quantitated by densitometry. For each transcript, translation in the absence of drug was set at 100%.

FIG. 5 is the chemical structure of Hoescht Dye H33258.

FIG. 6 is the chemical structure of Hoescht Dye H33342.

FIG. 7 is the nucleotide sequence and predicted secondary structure of H33258 aptamer H10, based upon the computer modeling program Mulfold. A Hoescht dye aptamer consensus sequence (UUAN₄₋₅UCU) was identified after 10 rounds of selection. The fixed primer binding regions are shown in plain print, selected bases are in bold, and the selected consensus sequence is indicated by outline print.

FIG. 8 is the nucleotide sequence and predicted secondary structure of H33258 aptamer H19, based upon the computer modeling program Mulfold.

FIG. 9 is a histogram summarizing data on the interaction of H10 and H19 aptamers with H33258, as indicated by percentage of total bound RNA eluted from an affinity column. Labeled aptamer (200,000 cpm of ³²P-UTP) was loaded onto a 0.25 ml H33258-Sepharose column. Each column was then washed sequentially with 6 ml binding buffer, 1 ml binding buffer containing 5 mM H33258, and 1 ml binding buffer containing 25 mM H33258. Fractions were collected and quantitated by scintillation counting.

FIG. 10. is a histogram summarizing SDS-PAGE densitometry data from in vitro translation experiments. RNA transcripts containing 0 (RSETA) or 2 copies of an H33258 aptamer (H2-RSETA) were translated in a wheat germ extract in the presence of ³⁵S-methionine and 0, 40 or 80 μM H33258. Protein products were subjected to SDS-PAGE and quantitated by densitometry. For each transcript, translation in the absence of drug was set at 100%.

FIG. 11 is a histogram summarizing data from in vivo expression experiments. H33258 aptamers H10 and H19 were cloned in tandem into the 5′ UTR of a β-galactosidase reporter gene (SVβ gal; Promega) to generate SVH2β gal. CHO cells were cotransfected with 1 μg SVβ gal or SVH2β gal and 1 μg of a luciferase expression vector (pGL3). Transfected cells were grown in the presence of 0, 5, or 10 mM H33342. Twenty-four hours after transfection, cell extracts were prepared, and β-galactosidase and luciferase activities were determined.

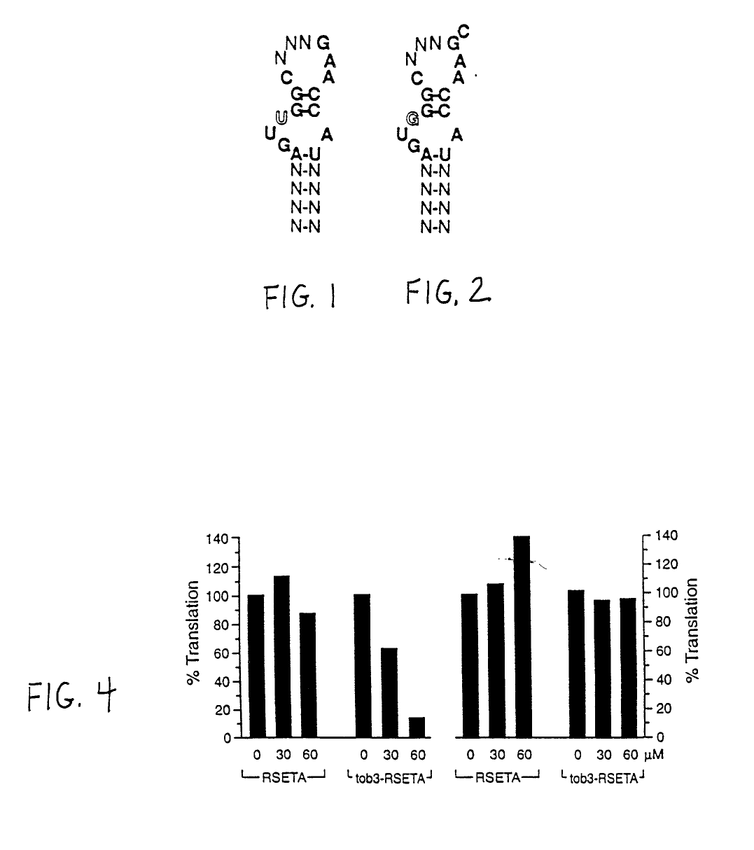

### DETAILED DESCRIPTION

Providing an Aptamer

Techniques for in vitro selection of aptamers that bind specifically to a particular cell-permeable molecule, i.e., ligand, are known in the art. Those techniques can be employed routinely to obtain an essentially unlimited number of apatmers useful in the present invention. Examples of publications containing useful information on in vitro selection of aptamers include the following: Klug et al., Molecular Biology Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996).

The basic steps in conventional in vitro selection of an aptamer are as follows. A random DNA pool is synthesized, i.e., a pool of DNA molecules having random nucleotide sequences. The random DNA pool is transcribed to produce a random RNA pool. The RNA pool is subjected to affinity chromatography. RNA molecules that bind specifically to the immobilized ligand are collected and reverse-transcribed into cDNA and amplified by PCR. The PCR-amplified products are transcribed into RNA. The process is repeated for as many cycles as necessary to yield a population of nucleic acid molecules that bind to the ligand with the desired affinity (and specificity). Individual nucleic acid molecules from the selected population are cloned and sequenced using conventional recombinant DNA technology. Such technology is described in numerous references, e.g., Sambrook et al., Molecular Cloning—A Laboratory Manual (2nd ed.), Cold Spring Harbor Laboratory Press (1989).

For any given cell permeable, small molecule (ligand), a potentially large number of different, useful aptamers can be isolated by one of ordinary skill in the art, using conventional techniques, without undue experimentation. The aptamers are empirically selected from a random pool of nucleic acid molecules by predictable selection methods. Therefore, it is not necessary to know in advance of the selection process what the nucleotide sequence of the aptamer will be.

The optimal length of the random nucleotide sequence in the aptamer length will vary, depending on factors including the size and shape of the ligand. Preferably, the length of an aptamer used in this invention is between 10 and 200 nucleotides. More preferably, the length is between 20 and 100 nucleotides.

Among the numerous aptamer-ligand pairs useful in this invention, aptamer-ligand binding affinities can vary widely. In general, the affinity is high enough to provide effective control of gene expression, but not so high as to make the aptamer-ligand binding effectively irreversible. Determination of whether a particular aptamer-ligand pair displays a suitable binding affinity is within ordinary skill in the art.

Incorporating the Aptamer

After isolation of an aptamer that binds the cell permeable molecule (ligand) with suitable affinity and specificity, the aptamer is incorporated into the 5′ UTR of a gene whose expression is to be controlled. The incorporation can be carried out, without undue experimentation, using conventional recombinant DNA technology.

The gene whose expression is to be controlled can be an endogenous gene or a transgene. When the gene is an endogenous gene, the aptamer can be incorporated into the 5′ UTR by known techniques of gene targeting, i.e., homologous recombination. When the gene is a transgene, preferably the aptamer is incorporated into the 5′ UTR by in vitro manipulation of the transgene or a DNA vector containing the transgene.

A gene controlled according to. this invention can be in a prokaryote or a eukaryote. The gene can be in an episome, e.g., a plasmid, or a genome, e.g., a mammalian chromosome. A transgene or gene targeting vector can be introduced into the living cell (that will be contacted with the cell permeable molecule), or a progenitor of the cell, by any suitable means. The suitable means will depend, at least in part, on the identity of the living cell. This is illustrated by the following non-limiting examples. If the living cell is a yeast cell, the transgene or gene targeting vector can be electroporated directly into the yeast cell or a progenitor of the yeast cell. If the cell is in a transgenic plant, the transgene or gene targeting vector can be introduced into regenerable plant tissue culture cells by electroporation, ti-plasmid, or microparticle bombardment. If the living cell is a cell in a transgenic, non-human mammal, the transgene or gene targeting vector can be microinjected into an embryonic cell that is used to produce the non-human mammal. If the cell is in vivo in a human receiving gene therapy, the transgene or gene targeting vector can be introduced into target cells of the human by any suitable gene therapy technique, e.g., a viral vector or injection of naked DNA.

Cell Permeable, Small Molecule

There is wide latitude in the choice of the cell permeable, small molecule used in this invention. The cell permeable, small molecule must bind an aptamer with suitable affinity and specificity. Whether a molecule will bind an aptamer with suitable affinity and specificity depends on factors including molecular size, shape and charge. Those of skill in the art will appreciate that the cell permeable molecule can be chosen first, and then used for in vitro selection of an aptamer that binds to it. Choosing a cell permeable, small molecule that is suitable for use in in vitro selection of an aptamer is within ordinary skill in the art.

Preferably, the cell permeable, small molecule displays low toxicity, so that unwanted biological side effects are minimized. When the cell containing the gene to be controlled is in vivo, the cell permeable, small molecule is chosen to have an in vivo persistence sufficient to allow an effective amount of the cell permeable, small molecule to reach and enter the cell.

In some embodiments of the invention the cell permeable, small molecule is a drug previously approved for use in humans. Using an approved drug can be advantageous, because information on safety, side effects, dosage, route of administration, pharmacokinetics, metabolism, clearance and other useful information is available. Preferred drugs are those that display mild pharmacological activities and minimal side effects.

It is not necessary, however, for the cell permeable, small molecule to be a drug. In preferred embodiments of the invention, the cell permeable, small molecule is pharmacologically inert (except for its activity in binding the aptamer according to this invention). Preferably, the cell permeable, small molecule is an organic compound. The design and synthesis of small, organic, cell permeable molecules useful in this invention are described, for example, in Amara et al., Proc. Natl. Acad. Sci. USA 94:10618-10623 (1997); and Keenan et al., Bioorganic & Medicinal Chemistry 6:1309-1335 (1998).

Formulating and Administering the Cell Permeable, Small Molecule

The cell permeable, small molecule can be formulated, individually or in combination, into pharmaceutical compositions by admixture with pharmaceutically acceptable nontoxic exipients and carriers. Such compositions can be prepared for use in parenteral administration, particularly in the form of liquid solutions or suspensions; for oral administration, particularly in the form of liquid, tablets or capsules; or intranasally, particularly in the form of powders, nasal drops, or aerosols.

The composition can be administered conveniently in unit dosage form and can be prepared by any of the methods known in the art. Such methods are described, for example, in Remington's Pharmaceutical Sciences (Mack Pub. Co., Easton, Pa., 1980).

Liquid dosage forms for oral administration include pharmaceutically acceptable emulsions, microemulsions, solutions, suspensions, syrups and elixirs. In addition to the active compound, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable depot forms are made by forming microencapusule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, 3) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. In solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Target Validation

The present invention can be used in “target validation” studies. The goal of target validation is to determine whether a particular gene is essential for the survival or growth of a particular type of cell, e.g., a bacterial pathogen. If a gene of interest is an essential gene, it (or its expression product) constitutes a potential drug target, which can be used for drug screening or rational drug design.

Target validation technology has previously relied on a conventional gene “knockout” approach. See, e.g., Arigoni et al., Nature Biotechnology 16:851-856 (1998). A disadvantage of the conventional gene knockout approach is that the gene is either present or absent, i.e., intermediate levels of expression of the gene of interest are not evaluated.

The present invention advantageously allows measurement of the effect of intermediate levels of expression of the gene of interest. For example, a 50% reduction in expression of an essential gene might be sufficient to cause the death of a microbial pathogen. Such information, now can be obtained readily through the use of this invention.

### EXAMPLES

The invention is further illustrated by the following examples. The examples are provided for illustration purposes only, and are not to be construed as limiting the scope or content of the invention in any way.

We demonstrated that bacteria expressing an aptamer to an aminoglycoside antibiotic are resistant to the cognate drug. This indicated that a small molecule-aptamer interaction occured in vivo. To regulate gene expression, aminoglycoside aptamers were inserted into the 5′ UTR of an mRNA, whose in vitro translation then became repressible by drug addition. To determine if a similar approach could work in vivo, we derived RNA aptamers for cell-permeable Hoechst dyes and inserted them into the 5′ UTR of a β-galactosidase reporter gene. Following transfection into mammalian cells, expression of the reporter gene was specifically inhibited by drug addition.

An initial 70 nucleotide RNA pool containing 31 random nucleotides was constructed essentially as described by Singh et al., Science 268:1173 (1995). Tobramycin or kanamycin A were covalently linked to CNBr-activated Sepharose 4B. Aminoglycosides (2 mmoles) were dissolved in coupling buffer (0.1 M NaHCO_{3,} 0.5 M NaCl, pH 8.3), then mixed with CNBr-activated Sepharose 4B (preswollen in 1 mM HCl) and incubated at 4° C. for 12-16 hours. The resin was then washed and remaining active groups blocked with 0.2 M glycine. Pre-selection columns were prepared with glycine alone.

The RNA pool (approximately 10¹⁵ individual sequences) was dissolved in selection buffer (50 mM Tris, pH 8.3, 250 mM KCl, 2 mM MgCl₂) heated to 80° C. for 3 minutes and cooled to room temperature. RNA was then loaded onto a pre-selection column (0.25 ml glycine-Sepharose) to remove RNAs that bound to the column, the resin, or glycine. Non-binding RNAs were eluted with two column volumes of selection buffer and immediately loaded onto a 0.5 ml aminoglycoside-Sepharose column. Columns were washed with 10 column volumes of selection buffer (selection rounds 1-5), 10 column volumes buffer with 5 mM competitor aminoglycoside (rounds 6-9), or 10 column volumes buffer with 10 mM competitor (rounds 10-14). The competitor aminoglycoside for tobramycin aptamer selection was kanamycin A and vice versa. In each round, bound RNA was eluted with 5 mM of the cognate aminoglycoside.

Eluted RNA was RT-PCR amplified using flanking primers. The PCR products were transcribed into RNA with T7 RNA polymerase and purified by polyacrylamide gel electrophoresis. Pools were subcloned into the plasmid pBlueScript (Stratagene) and sequenced after rounds 10, 12, and 14. Isolation of H33258 aptamers was carried out in a similar manner, with the following exceptions. H33258 was covalently linked to epoxy-activated Sepharose 6B. The ligand solution was mixed at 37° C. for 16 hours. The resin was then washed and excess active groups were blocked with 1 M ethanolamine (pH 10). Pre-selection columns were prepared with ethanolamine alone. H33258 selection buffer contained 50 mM Tris pH 7.3, 200 mM KCl, 2 mM MgCl₂.

In selection rounds 1-6, columns were washed with 20 column volumes of selection buffer and eluted with 2 column volumes of 10 mM H33258. In selection rounds 7-10, columns were washed with 20 column volumes buffer and 20 column volumes 10 mM benzimidazolepropionic acid (in selection buffer) before elution.

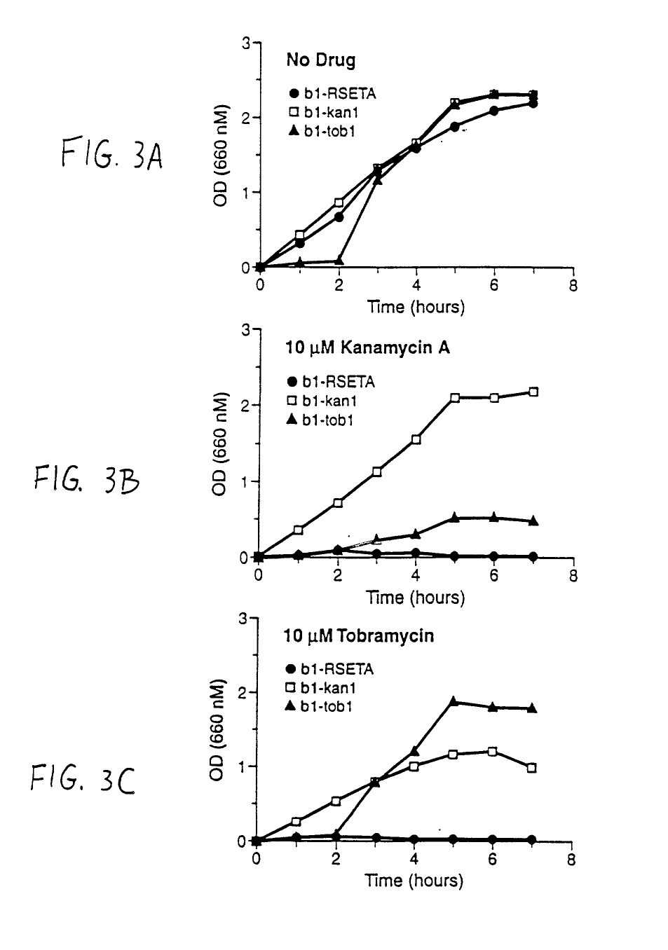

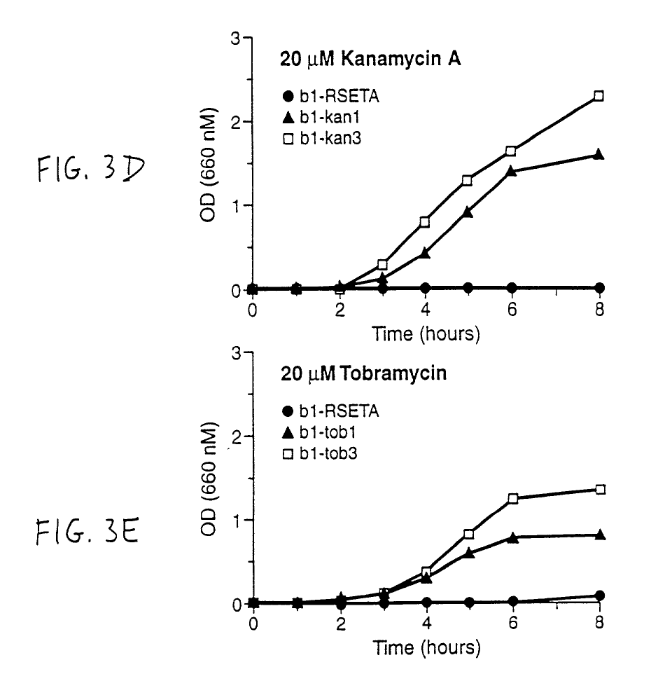

FIG. 1A shows the consensus sequences and secondary structures of our kanamycin A and tobramycin aptamers, which differ at only two of fourteen bases. As an initial test for the ability of these aptamers to function in vivo, we asked whether following expression in E. coli the aptamer would sequester the cognate antibiotic thereby conferring a specific drug-resistant phenotype. Toward this end, one or three copies of the kanamycin A (kan) or the tobramycin (tob) aptamer were cloned into the T7 RNA polymerase-driven expression vector PRSETA (Invitrogen), and transformed into a bacterial strain containing an IPTG-inducible T7 RNA polymerase. Bacterial strains were grown in liquid culture overnight and then diluted into antibiotic-containing medium. In the absence of drug, bacterial strains expressing no aptamer (bl-RSETA), the kanamycin aptamer (bl-kan1), or the tobramycin aptamer (bl-tob1) grew similarly (FIG. 3A). In the presence of 10 mM kanamycin A, bl-kan1 grew to saturation, whereas growth of bl-RSETA and b-tob1 was neglible (FIG. 3B). In the presence of 10 mM tobramycin, bl-tob1 grew to saturation, and bl-kan1 also grew to a sub-saturating level (FIG. 3C). The partial-resistance of bl-kan1 to tobramycin (our unpublished data). FIGS. 3D and 3E show that increasing the number of aptamers in the expression vector from one to three, enhanced growth in the presence of antibiotic. None of the strains exhibited increased resistance to unrelated antibiotics. Collectively, these results indicate that a specific drug-resistant phenotype can be conferred by expression of an aminoglycoside aptamer, demonstrating the occurrence and specificity of a small molecule-aptamer interaction in vivo.

Based upon the in vitro results, we next designed experiments to investigate whether small molecule aptamers could be used to regulate gene expression in vivo. We designed these experiments in view of the fact that eukaryotic translation initiation typically involves 5′-to-3′ scanning from the 5′-m⁷G cap to the start codon (Kozak, Ann. Rev. Cell Biol. 8:197 (1992); Sachs et al., Cell 89:831 (1997)), and binding of a protein between the cap and start codon can repress translation, presumably by blocking either scanning or the ribosome-mRNA interaction (Stripecke et al., Mol. Cell. Biol. 14:5898 (1994); Paraskeva et al., Proc. Natl. Acad. Sci. USA 95:951 (1998)). These considerations prompted us to test whether the presence of a small molecule-aptamer complex within the 5′ UTR would repress translation in an analogous fashion.

A test mRNA was constructed containing three copies of the tob aptamer inserted in the 5′ UTR of RSETA (tob3-RSETA). In vitro translation reactions were performed in the presence of 0, 30 or 60 μM tobramycin or kanamycin A.

In vitro transcription reactions contained 5 μg pRSETA (or RSET derivative), 0.5 mM m⁷G(5′)G, 0.5 mM ATP, CTP, UTP, 0.05 mM GTP, 10 mM DTT and 40 U T7 RNA polymerase in 50 μl of a solution of 40 mM Tris-HCl pH 7.5, 6 mM MgCl2, 2 mM spermidine, 10 mM NaCl. Following incubation for 1 hour at 37° C., RNA was purified by phenol:chloroform extraction, ethanol precipitation and resuspended in 30 μl H₂O. Translation reactions were carried out in 10 μl containing 5 μl wheat germ extract, 0.8 μl 1 mM amino acid mixture (minus methionine), 2 μl of RNA transcript (described above), 0.5 μl [³⁵S] methionine (1200 Ci/mmole) and 0-80 μM drug. Reactions were incubated at 25√ C. for 15 minutes and terminated by addition of 2X sample loading buffer. Translation products were separated by electrophoresis on an 18% polyacrylamide gel, visualized by autoradiography, and quantitated by densitometry.

Translation of the control RSETA mRNA was unaffected by all concentrations of tobramycin or kanamycin tested. Addition of tobramycin inhibited in vitro translation of the tob3-RSETA mRNA in a dose-dependent fashion (FIG. 4). In vitro translation of the tob3-RSETA MRNA was not inhibited by comparable concentrations of kanamycin A, which is not recognized by the tob aptamer.

Our results indicated that small molecule-aptamer interactions occur faithfully in vivo (FIGS. 3A-3E). The results summarized in FIG. 4 showed that in a cell-free system a small molecule can be used to regulate translation through a cis-acting aptamer. We therefore reconfigured the system for regulating gene expression in vivo. Because aminoglycosides were known to be relatively impermeable to the plasma membrane, to be cytotoxic, and at elevated concentrations to have a general inhibitory effect on translation, we elected to use a different cell-permeable small molecule as the translation regulator.

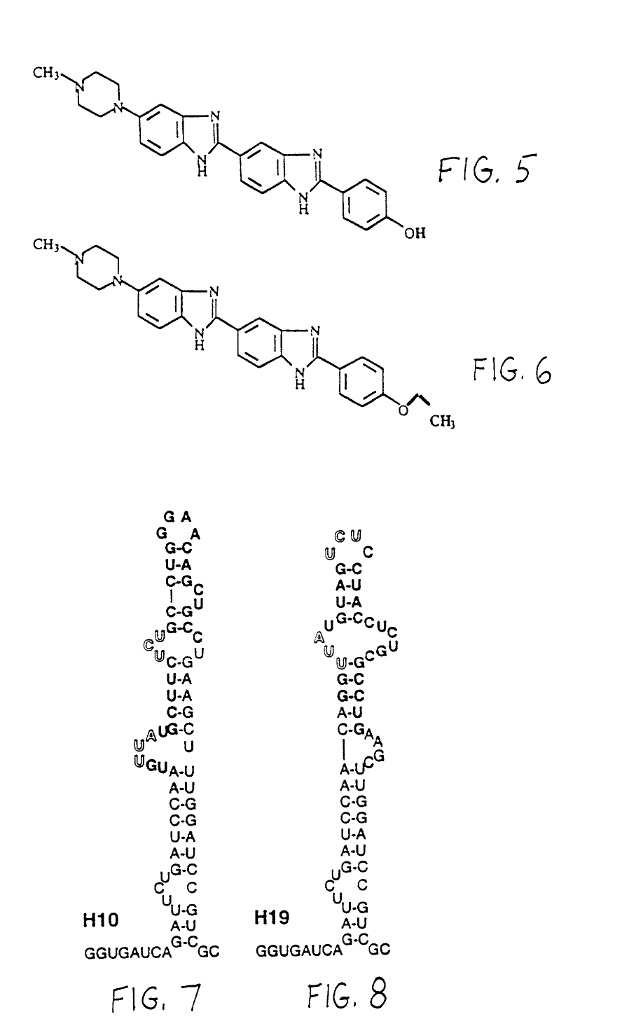

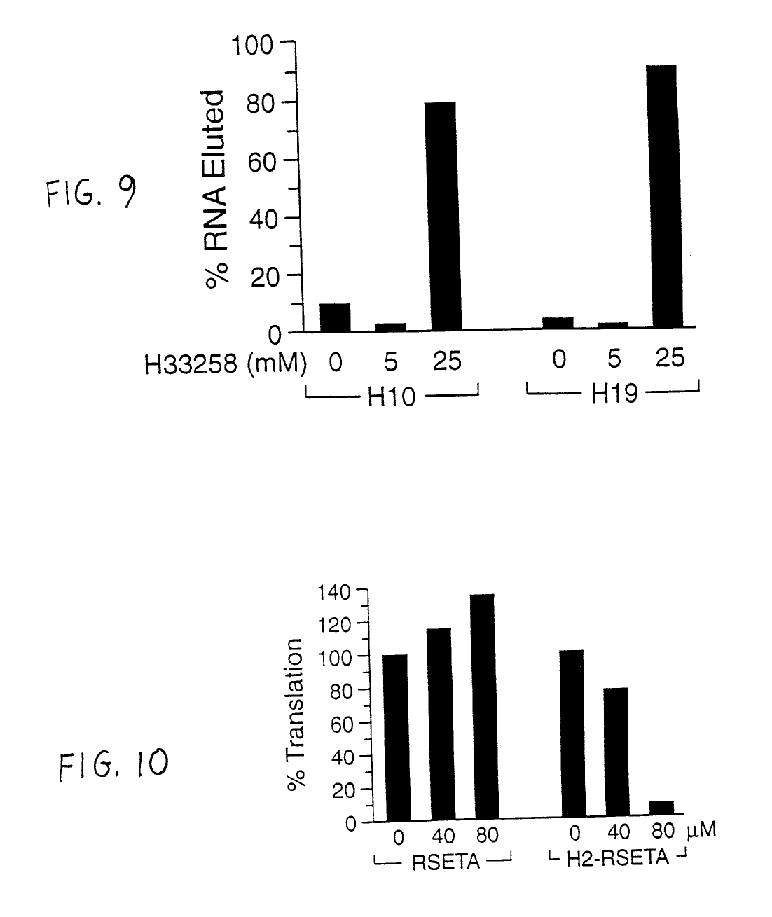

We chose the Hoeschst dye 33258 (H33258) and the closely related drug H33342 (FIGS. 5 and 6), because they were known to be relatively non-toxic and cell-permeable (Uphoff et al., Curr. Opin. Struct. Biol. 6:281 (1996)). We isolated RNA aptamers that bound specifically to H33258 by affinity chromatography on a column containing H33258 covalently attached to an epoxy-activated sepharose resin through a single hydroxyl group. FIGS. 7 and 8 show the sequences and secondary structures of two of these aptamers, H10 and H19, isolated after 10 rounds of selection. H10 and H19 bound to an H33258 affinity-column and required a relatively high concentration (25 mM) of free H33258 for elution (FIG. 9). H10 and H19 bound H33258 and the closely related H33342 comparably (data not shown).

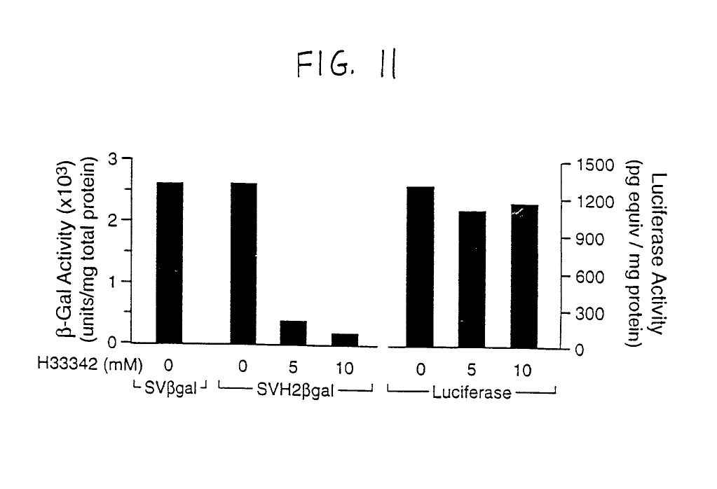

To demonstrate that the H33258-aptamer could be used to regulate translation, one copy of H10 and H19 were inserted in tandem into the 5′ UTR of RSETA. Addition of H33258 inhibited in vitro translation of H2-RSETA, but not the control RSETA, in a dose-dependent fashion (FIG. 10).

To test whether this small molecule-aptamer interaction could be used to control gene expression in vivo, one copy of H10 and H19 were inserted into the 5′ UTR of a mammalian β-galactosidase expression plasmid SVβ Gal (Promega), generating the construct SVH2β gal. CHO cells were cotransfected with SVH2β Gal or as a control the parental vector, SVβ Gal, and a luciferase reporter gene to provide an internal control. Following transfection, cells were grown for 24 hours in the presence of 0, 5 or 10 μM H33342 and analyzed for β-galactosidase and luciferase activities. In these experiments, H33342, rather than H33258, was used because it is approximately ten-fold more cell-permeable.

In the absence of drug, two H33258 aptamers in the 5′ UTR had no effect on gene expression (compare SVβ gal and SVH2β gal) (FIG. 11). This was consistent with the in vitro translation data shown in FIG. 10. Expression of the luciferase reporter (FIG. 11) and the parental expression vector SVβ Gal (data not shown) were not inhibited by 0, 5 or 10 μM H33342. H33342 reduced β-galactosidase activity from SVH2β Gal greater than 90% in a dose-dependent fashion. These results indicated that inhibition by H33342 is dependent upon the presence of an appropriate RNA aptamer in the 5′ UTR, and that the small molecule-aptamer translation switch works both in vitro and in vivo.

H33258 aptamers, H10 and H19, were cloned in tandem into the 5′ UTR of a β-galactosidase reporter gene (SVβ gal, Promega) to generate SVH2β gal. CHO cells were cotransfected with 1 μg SVβ gal or SVH2β gal and 1 μg of a luciferase expression vector (pGL3). Transfected cells were grown in the presence of 0, 5 or 10 mM H33342. 24 hours post-transfection cell extracts were prepared and β-galactosidase and luciferase activities were determined.

Other embodiments are within the following claims.

We Claim:

1. A method for controlling the expression of a gene in a living cell, comprising contacting the 5′ untranslated region of an RNA in the cell with a cell permeable, small molecule.

2. A method for controlling expression of a gene, comprising:
providing an aptamer that binds specifically to a cell permeable, small molecule;
incorporating the aptamer into a region of a gene, which region encodes a 5′ untranslated region of an RNA;
contacting the cell-permeable, small molecule with a cell that contains the gene, so that the cell-permeable, small molecule enters the cell and controls expression of the gene.

3. The method of claim 2, wherein the cell permeable, small molecule binds specifically to the aptamer sequence in the 5′ untranslated region of RNA transcribed from the gene.

4. The method of claim 2, wherein the gene is an endogenous gene.

5. The method of claim 2, wherein the gene is a transgene.

6. The method of claim 2, wherein the cell is a prokaryotic cell.

7. The method of claim 2, wherein the cell is a eukaryotic cell.

8. The method of claim 7, wherein the eukaryotic cell is a mammalian cell.

9. The method of claim 8, wherein the mammalian cell is in vivo.

10. The method of claim 9, further comprising administering the cell permeable, small molecule to the mammal topically, parenterally, orally, vaginally, or rectally.

11. The method of claim 2, wherein the cell permeable, small molecule is an organic compound.

12. A gene comprising an aptamer sequence incorporated into a region of a gene that encodes a 5′ untranslated region of an RNA.

13. A transgenic cell comprising an aptamer incorporated into a region of a gene that encodes a 5′ untranslated region of an RNA.

14. The cell of claim 13, further comprising an RNA transcript containing the aptamer in the 5′ untranslated region of the RNA transcript.

15. The cell of claim 14, further comprising a cell permeable, small molecule that binds specifically to the aptamer.

16. A bacterial resistance marker comprising an aptamer sequence operably linked to a bacterial expression control sequence.

17. A method for determining whether a gene of interest is essential for the survival or growth of a cell, comprising:
structurally disrupting or deleting an endogenous gene of interest in the cell;
providing an aptamer that binds specifically to a cell permeable, small molecule;
incorporating the aptamer into a region of the gene of interest in vitro, which region encodes a 5′ untranslated region of an RNA, thereby producing a controllable gene of interest;
introducing the controllable gene of interest into the cell, thereby producing a test cell;
contacting the cell-permeable, small molecule with the test cell, so that the cell-permeable, small molecule enters the test cell and controls expression of the controllable gene of interest.
